(19) United States
(12) Patent Application Publication
(10) Pub. No.: US 2003/0185850 A1
(43) Pub. Date: Oct. 2, 2003

(54) Protective peptides neurotoxin of C. botulinum

(76) Inventor: Mark Dertzbaugh, Fairfied, PA (US)

Correspondence Address: Elizabeth Arwine, Office of Command Judge Advocate, HQ. USAMRDC, Department of the Army, Fort Detrick, Frederick, MD, 21702-5012, US

(21) Appl. No.: 09/917,791

(22) Filed: Jul. 31, 2001

Related U.S. Application Data
(63) Continuation-in-part of application No. 08/446,114, filed on May 19, 1995, now Pat. No. 6,287,566.

(51) Int. Cl. A61K 39/08; C07K 14/33

(52) U.S. Cl. 424/190.1; 424/239.1; 530/350

(57) ABSTRACT

Vaccines to protect from neurotoxins of C. botulinum have been developed. Trucated BoNT/A proteins of about 15-30 kDa in size produced immune responses that provided protection from neuronal damage by botulinum neurotoxins.

### FIELD OF THE INVENTION

This invention relates to immunization against toxic effect of neurotoxins of Clostridium bolutinum. Protective epitopes of the heavy chain of the neurotoxin of C. botulinum have been discovered. The invention also relates to preparation of protective immunotoxins of C. botulinum.

### BACKGROUND OF THE INVENTION

Botulinum neurotoxin (BoNT) is one of the most potent toxins known to man. Ingestion or inhalation of toxin inhibits neurotransmitter release from synaptic vesicles, resulting in neuroparalysis and death. Seven serologically distinct forms of neurotoxin are produced by Clostridium botulinum. The toxin is synthesized as a 150 kDa precursor that is proteolytically nicked into two subunits. The light (L) chain, associated with the toxicity of BoNT, must be internalized in the cell in order to inhibit neurotransmitter release. It is linked by a disulfide bond to the heavy (H) chain, which mediates binding of the toxin to receptors located on the surface of the nerve cell. Although the heavy chain is required for BoNT to productively bind and enter the target cell, it is not toxic by itself.

The current pentavalent toxoid vaccine for botulism is composed of formalin-inactivated holotoxin. Although effective, this vaccine is difficult to manufacture. Furthermore, extensive treatment with formalin is required to inactivate the toxin. Prolonged treatment with formalin can affect the immunogenicity of protein antigens, and this may explain why certain lots of toxoid have been poorly immunogenic in the past.

There are several approaches that can be used to construct a new vaccine. One approach would be to express a non-toxigenic mutant of BoNT/A, as has already been done for other toxins. The advantage of this approach is that the immune response elicited by the modified protein would most closely approximate the response elicited by the native toxin, because almost all of the native protein structure would still be intact. However, high level expression of the C fragment of tetanus toxin (TeTx) could not be achieved in E. coli when the native clostridial gene sequence was used. Based on this information, expression of BoNT might be predicted to be difficult, as well. Another approach is to construct a synthetic peptide-based vaccine. The advantage of this approach is that large quantities of synthetic peptide can be easily manufactured for use in a vaccine. However, studies with IvAbs have indicated that many of the neutralizing epitopes located on BoNT are conformationally sensitive. This suggests that a peptide-based vaccine may not necessarily be able to induce neutralizing antibody responses due to its lack of conformational epitopes. A genetically engineered vaccine for botulism would eliminate many problems, since it could be expressed in a recombinant host at high levels and would not require treatment with formalin before incorporation into a vaccine.

Recent developments have made the construction of a genetically engineered BoNT vaccine possible. The gene for BoNT serotype A (BoNT/A) has been cloned and sequenced (Binz, et al., J. Biol. Chem. 265:9153-9158.(1990), and the minimum length of the light chain needed to retain neurotoxicity has been defined (Kurazono, et al., J. Biol. Chem. 267:14721-14729 (1992)). While construction of such a vaccine is feasible, there has not been a systematic attempt to identify the domain(s) of BoNT/A that would be required to elicit protective immunity. Immunization with a fragment corresponding to the C-terminal half of the heavy chain (H_{C}) has been shown to stimulate protective immunity, but more definitive identification of sequences that elicit protective immune response had not Previously been described. Monoclonal antibodies directed against either light chain or heavy chain determinants had been shown to provide some passive protection to mice against a lethal exposure to BoNT, indicating that protective epitopes may exist on either chain. However, many of these epitopes appear to be conformationally sensitive, which suggests that mapping their location by using synthetic peptides may be unproductive due to their lack of tertiary structure.

### SUMMARY OF THE INVENTION

It is the purposes of this invention to provide methods for developing vaccines to protect from neurotoxins of C. botulinum. The methods used to identify specific sequences consisted of amplifying and cloning overlapping segments of the BoNT/A gene.

These segments are then expressed in suitable vectors such as E. coli to produce truncated BoNT/A proteins of about 15-30 kDa in size. The truncated proteins are purified by appropriate methods such as SOS-PAGE. The invention is exemplified using two particularly protective regions from the heavy chain of the type A C. botulinum toxin. The peptides giving rise to protective antibodies may be fused to other peptides that act as adjuvants to increase antigenicity. Such fusion proteins may be produced by recombinant technology using plasmids containing hybrid genes for expression of the desired fusion proteins.

### DETAILED DESCRIPTION OF THE INVENTION

It is the purpose of this invention to identify and provide 20 immunogenic polypeptides which give rise to protective antibodies against botulism. Compositions containing the subject polypeptides in pharmaceutically acceptable carriers are useful as vaccines and as diagnostic agents to identify protective antibodies. 25 The location of protective domains was identified, and those domains were produced by expressing fragments of BoNT/A in E. coli and then evaluating each for its protective efficacy. Using this approach, fragments of the BoNT/A gene were expressed that were of sufficient size to still possess some tertiary conformation, but that would greatly reduce the amount of the toxin utilized. By overlapping the regions of the BoNT/A protein being expressed, it was possible to minimize the possibility that a locally encoded epitope was accidently interrupted. The advantage of this approach is that the fragments were sufficiently small to be nontoxic. However, it is possible that not all protective determinants may have been encoded by these fragments.

It was possible to express fragments of the BoNT/A gene at high levels in E. coli by using an inducible T7 expression system. It was not predictable that, in contrast to some of the problems encountered with expression of the C fragment of tetanus toxin (TeTx), this could be done for purposes of making a protective vaccine against botulism. Some difficulty encountered which was related to the fact that clostridial toxin is encoded by codons that are rarely used by E. coli. Unexpectedly, this problem with the DNA sequence naturally encoded by Clostridium did not present the barrier that might have been expected. The possibility for expression of these proteins may be due to the size of the BoNT/A proteins encoded. The TeTx proteins being expressed in E. coli were two to three times larger than the BoNT/A proteins expressed as disclosed herein. The smaller size of the BoNT/A transcripts may have permitted E. coli to translate them more efficiently. However, the truncated BoNT/A proteins were expressed primarily in the form of insoluble inclusion bodies. Insertion of the BoNT/A gene fragments into the plasmid vector pMTD74 resulted in expression of a BoNT/A protein fused to the A2 peptide of cholera toxin (CtxA2) at its C-terminus. These fragments were fused to CtxA2 to associate noncovalently with the B subunit of cholera toxin (CtxB). Fusion of antigens to CtxB was shown to improve their immunogenicity when administered by mucosal routes of immunization (Dertzbaugh, et al., Infect. Immun. 61:48-55 (1993)). Hence, CtxB is used as a delivery system with these fragments of BoNT/A as part of a mucosally administered vaccine for botulism.

The ability of the BoNT/A fragments to induce an antibody response was affected by the antigen preparation used for immunization. Effective production of antibody to BoNT/A was inadequate when the crude lysates were used for immunization, even though they contained relatively large amounts of BoNT/A-specific protein. For this reason, immunization was performed again with highly enriched preparations of the BoNT/A proteins. Unlike the crude form of the antigen, the purified form was able to elicit BoNT/A-specific antibody whilst being well tolerated by the animals. The poor immunogenicity of the crude lysates may have been due to saturation of the antigen-presenting cells with other antigens present. It is possible that by purifying the BoNT/A proteins, other immunodominant antigens were removed which could have been competing for uptake and presentation to lymphocytes by the antigen-presenting cells.

Preparative SDS-PAGE was used to purify the BoNT/A fragments for several reasons. First, most of the BoNT/A protein present in the lysates were in the form of inclusion bodies that had to be solubilized before purification. SDS easily solubilized the BoNT/A proteins. Second, this method can be used to purify all of the fragments, regardless of their size or composition. Furthermore, the size range of the BoNT/A proteins permitted them to be separated from most of the other proteins present in the lysates. One potential disadvantage of using such a denaturing method is. that the purified BoNT/A proteins may not have completely resumed their native conformation, resulting in the loss of some epitopes. The BoNT/A proteins should have been able to refold when the SDS was removed from the antigen preparations before immunization.

Hybrid gene fusion proteins may also be produced to increase protective immune response. For example, DNA sequences which encode desired antigenic polypeptides may be fused to DNA sequences which encode non-toxic peptides of other organisms such as cholera. U.S. Pat. No. 5,268,276 to Holmgren, et al., which is incorporated herein in its entirety by reference, discloses a means of producing an appropriate fusion gene to produce fusion proteins containing the immunogenic peptides of botulism.

Both BoNT peptides and fusion proteins containing BoNT amino acid sequences may be administered by mouth. Antigenic fusion proteins containing sequences of cholera subunits are useful for administration orally or to the mucosa (for example intranasally). The fusion proteins may be lyophilized and inhaled from a vial for administration.

Compositions containing the BoNT peptides in pharmaceutically acceptable carriers may also be administered parenterally. Preferred parenteral routes include intracutaneous or subcutaneous or intramuscular injection. Any of the compositions may contain, additionally, adjuvants such as alum or Freund's adjuvant. While the invention has been exemplified using the peptides of C. botulinum, serotype A, analogous polypeptides sequences of other serotypes can be made in the manner described herein. A cocktail of polypeptides from various serotypes may be administered to provide broad protection against toxins of C. botulinum serotypes.

### Materials and Methods

Construction of the BoNT/A gene fragments. The polymerase chain reaction (PCR) was used to amplify and clone overlapping fragments of the BoNT/A gene. Primers used to amplify each fragment are listed in Table 1. The primers were designed to include unique flanking restriction sites on the 5′ and 3′ ends of each amplified fragment in order to permit its insertion into the expression vector. Plasmids pCBA2, pCBA3, and pCBA4 encoding large overlapping regions of the BoNT/A gene and flanking DNA were used as template DNA (Thompson, et al., Eur. J. Biochem. 73-81 (1990)). Amplification was performed using Vent DNA Polymerase (New England Biolabs, Beverly, Mass.). The reaction mixture was prepared according to the manufacturer's directions, and consisted of 100 ng/μl forward primer, 100 ng/μl reverse primer, and 10 ng/μl template. Each reaction was subjected to 25 cycles of amplification in a DNA thermocycler according to the following parameters: melting temperature, 94° C. for 1 min; annealing temperature, 45° C. for 1 min; extension temperature, 72° C. for 1 min. The amplified DNA was digested with the appropriate restriction enzymes and then was ligated into the expression vector pMTD74.

TABLE 1

PCR Primers

| | | |
|---|---|---|
| n.t.^{a} | Direction^{c} | Sequence |

| | |
|---|---|
| 367-741 | F: 5′-ATATGGAATTCGTTAATAAACAATTTAATTATAAAGATCC-3′ |
| L₄₋₂₁₈^{b} | R: 5′-AGTATCGTCGACTTTTAATTCTGTATCTATTGTACTTCCACC-3′ |
| 732-1170 | F: 5′-GATACAGAATTCAAAGTTATTGATACTAATAG-3′ |
| L₁₂₆₋₂₇₁^{b} | R: 5′-CTTTGCGTCGACTCCCCCAAATGTTCTAAGTTCC-3′ |
| 1126-1750 | F: 5′-GGGTTAGAATTCAGCTTTGAGGAACTTAGAACATTTGGG-3′ |
| L₂₅₇₋₄₆₅^{b} | R: 5′-AGGACTGTCGACCAAGTCCCAATTATTAACTTTGATTGATAAATC3′ |
| 1720-2340 | F: 5′-TTAAATGAATTCTCAATCAAAGTTAATAATTGGGAC-3′ |
| H₄₅₅₋₆₆₁^{b} | R: 5′-CTCTGGGTCGACTTCTAACAGAATAACAGCTCC-3′ |
| 2150-2780 | F: 5′-GAAGTAAGAGCTCTGGATAAAATTGCGGATATAAC-3′ |
| H₆₃₀₋₈₀₈^{b} | R: 5′-TAACCGGTCGACACCATAAGGGATCATAGAG-3′ |
| 2695-3175 | F: 5′-GCTATGATTAATATAAATAAATTTTTGAATCAATGC-3′ |
| H₇₈₀₋₉₃₉^{b} | R: 5′-AGTACTAAGCTTTTCATACATACTATTATATACAATAGC-3′ |
| 3100-3530 | F: 5′-AAAAATAGAGCTCAATTATTTAATTTAGAAAGTAG-3′ |
| H₉₁₅₋₁₀₅₉^{b} | R: 5′-ACCATCGTCGACAAACATTATATTATTACTAGC-3′ |
| 3301-3726 | F: 5′-TATGGTGAATTCATCTGGACTTTACAGGATACTCAGG-3′ |
| H₉₈₂₋₁₁₂₃^{b} | R: 5′-ATTTACGTCGACATATTTATTTGGATC-3′ |
| 3590-4020 | F: 5′-GATAAGGAATTCAATGAAAAAGAAATCAAAG-3′ |
| H₁₀₇₈₋₁₂₂₀^{b} | R: 5′-CTTCATGTCGACTACTTGACTTAGATTTCC-3′ |
| 3806-4223 | F: 5′-AACATTGAATTCAATTCAAGTTTGTATAGGGGG-3′ |
| H₁₁₅₀₋₁₂₈₉^{b} | R: 5′-TCCATCGTCGACAGGAATAAATTCCCATGAGCTACC-3′ |

^{a} Nucleotide sequence number designation based on EMBL/Genbank™ accession file X52066.
^{b} Amino acid residue number of the light (L) chain and the heavy (H) chain.
^{c} F, forward primer; R, reverse primer.

Bacterial strains and plasmids. Plasmids constructed are listed in Table 2. All plasmids were transformed by the CaCl₂-heat shock method (See Morrison, D. A., J. Bacteriol. 132:349-351(1977)) into E. coli strain HMS174(DE3) (Campell, et al., Proc. Natl. Acad. Sci., U.S.A., 75:2276-2280 (1978)). Bacterial strains were grown at 37° C. in M-9 medium in accord with the methods of Miller (Miller, J. H., Experiments in Molecular Genetics, Cold Spring Harbor Laboratory, Cold Spring Harbor (1972)) supplemented with 10 g of Casamino Acids (Difco Laboratories, Ann Arbor, Mich.) per liter, 20 μg of leucine per ml, 20 μg of proline per ml, 2 μg of thiamine per ml, 50 μg of ampicillin per ml, and 25 μg of rifampicin per ml. Plasmid pMTD74 was used to express the BoNT/A fragments in E. coli. It was derived from the T7 translation vector pET-8c (Studier, et al., Methods in enzymology, Academic Press, New York (1990)) and has a multiple Cloning site encoding unique restriction sites. This plasmid was used to express fragments of the BoNT/A gene in E. coli. It encodes a gene for ampicillin (Ap) resistance and a ColE1 origin of replication (ORI). Transcription is initiated from the T7 promoter and is terminated by the Tø transcriptional terminator (t.t.). Proper in-frame insertion of DNA within the multiple cloning site (MCS) results in expression of protein fused to the A2 peptide of cholera toxin (CtxA2). (Lockman, et al., J. Biol. Chem. 258:13722-13726 (1983)). The MCS contains several unique restriction sites, and is shown in more detail below the plasmid map. Translation is initiated by the Shine-Delgarno (S.D.) sequence located upstream of the initiator methionine encoded by the NcoI site. Transformation of this plasmid into the lysogenic E. coli strain HMS174(DE3) permits inducible expression of protein from the T7 promoter. T7 RNA polymerase is required for initiation of transcription from the T7 promoter, and this protein is inducibly expressed in HMS174(DE3) by using isopropyl-beta-D-thiogalactopyranoside (IPTG).

TABLE 2
Bacterial strains and plasmids

| E. coli | | | |
|---|---|---|---|
| Strain | Plasmid | Comments | References |
| | pMTD74 | | |
| HMS174 | | F⁻ hsdR(r_{K}⁻ m_{K}⁺) recA rif^{R} | Campbell* |
| HMS174(DE3) | | T7 expression host | Studier** |
| MTD103 | pMTD89 | BoNT/A L₄₋₁₂₈ | |
| MTD143 | pMTD143 | BoNT/A H₄₅₅₋₆₆₁ | |
| MTD145 | pMTD130 | BoNT/A H₇₈₀₋₉₃₉ | |
| MTD150 | pMTD74 | HMS174(DE3) host for | |
| | | background control | |
| MTD164 | pMTD162 | BoNT/A H₉₈₂₋₁₁₂₃ | |
| MTD165 | pMTD163 | BoNT/A H₁₁₅₀₋₁₂₈₉ | |
| MTD191 | pMTD186 | BoNT/A L₁₂₆₋₂₇₁ | |
| MTD193 | pMTD188 | BoNT/A H₁₀₇₈₋₁₂₂₀ | |
| MTD196 | pMTD187 | BoNT/A L₂₅₇₋₄₆₅ | |
| MTD203 | pMTD195 | BoNT/A H₉₁₅₋₁₀₅₉ | |
| MTD210 | pMTD148 | BoNT/A H₆₃₀₋₈₀₈ | |

*Campbell, et al., Proc. Natl. Acad. Sci., U.S.A. 75:2276-2280 (1994)
**Studier, et al., Methods in Enzymology, Academic Press, New York (1990)

Analysis of protein expression. Transformants were screened for protein expression by immnunoblotting analysis, Individual coIonies were grown at 37° C. in 2 ml of M-9 medium to an optical density of 0.8-1.0 at 660 nm. Expression was induced by adding IPTG to a concentration of 0.25 mM. After induction, cultures were incubated at 37° C. for 2 h before harvesting. The cells were pelleted in a microfuge tube and resuspended in 100 μl of sample loading buffer containing 1% sodium dodecyl sulfate (SDS) and 0.2 M 2-mercaptoethanol. The samples were boiled for 5 min and then separated by discontinuous SDS-polyacrylamide gel electrophoresis (PAGE) (Lammeli, U. K., Nature 227:680-685 (1970)). The proteins were transferred to nitrocellulose sheets using a semi-dry electroblotter (Integrated Separation Systems, Hyde Park, Mass,) and then stained for the presence of BoNT/A-specific protein using horse antiserum to BoNT/A. Strain MTD150 was prepared as described above and used as a background control. Purified BoNT/A (Sigma, St. Louis, Mo.) was included in each gel as a positive control.

Cell fractionation. Bacterial strains encoding the truncated BoNT/A proteins were grown in M-9 medium and were induced to express protein as described above. Cells were pelleted by centrifugation at 3,000× g for 10 min. The pellet was suspended in lysis buffer containing 1 mg/ml lysozyme, 50 mM Tris, 50 mM EDTA, and 20% sucrose (pH 8.0) and was incubated at 37° C. for 30 min. To ensure complete lysis, the cell suspension was subjected to two cycles of rapid freeze-thaw. MgSO₄ was added to the lysate to a concentration of 20 mM, DNase (Sigma) and RNase (Sigma) were added to a concentration of 0.01 mg/ml each, and then the lysate was incubated at 37° C. for 30 min. The lysate was clarified by centrifugation at 3,000× g for 10 min. The clarified lysate was centrifuged at 20,000× g for 30 min at 4° C. and the resulting pellet was dissolved in sample loading buffer. The sample was boiled for 5 min and stored at −20° C. before use.

Purification of BoNT/A proteins. The truncated BoNT/A proteins were purified by preparative SDS-PAGE with a Model 491 Prep Cell (Bio-Rad, Richmond, Calif.). The percentage of acrylamide used in the resolving gel was adjusted to maximize the separation of the protein of interest. Separation was typically performed at 12 watts constant power with a 37-mm diameter tube gel. The length of the stacking and resolving gels were 2 cm and 10 cm, respectively The eluate was collected at a flow rate of 0.75 ml/min as 4-ml fractions. Aliquots of the fractions were separated by analytical SDS-PAGE and stained with Coomassie blue to visualize total protein. in some cases, a duplicate gel was transferred to nitrocellulose and analyzed for immunoreactivity to polyclonal horse antiserum to BoNT/A. Fractions containing truncated BoNT/A protein were pooled and concentrated by ultrafiltration (Amicon, Danvers, Mass.). The concentrated protein was passed through a column containing Extracti-Gel™ D resin (Pierce, Rockford, Ill.) to remove any remaining SDS. The protein was subjected to extensive diafiltration in buffer containing 120 mM NaCl, 2.7 mM KCl, 10 mM phosphate buffer (pH 7.4), 20% glycerol (v/v), and 5 mM EDTA. Each protein preparation was examined by Coomnassie staining and immnunoblotting analysis for its composition and for the presence of BoNT/A-specific protein. Protein concentrations were determined by the BCA assay (Pierce). The protein preparations were aliquoted and stored at −70° C. before use.

Immunization and challenge. The protocol used in this study was approved by the USAMRIID Institutional Animal Care and Use Committee. Female CB6F1 mice (Jackson Laboratory, Bar Harbor, Me.), 4-6 weeks old, were provided food and water ad libitum. The mice were immunized with 10 μg of BoNT/A-specific protein suspended in adjuvant emulsion (Ribi Immunochem, Hamilton, Mont. ). Some mice were immunized with saline emulsified in adjuvant for use as negative controls. For comparison, some mice were immunized with pentavalent toxoid. The mice were immunized i.p. four times at 2-week intervals. One week after the last immunization, the mice were bled and the serum was analyzed by immunoblot for the presence of chain-specific antibody. Two weeks after the last immunization, each mouse was challenged i.p. with 2 lethal doses of BoNT/A (2 MIPLD₉₉) . Four days after challenges the mice were scored for survivors.

Immunoblotting analysis. BoNT/A was separated by SDS-PAGE on a 10% gel and then transferred to nitrocellulose using a semi-dry electroblotter. The nitrocellulose was blocked and loaded into a Multi-Screen immunoblotting apparatus (Bio-Rad). Pooled serum from each group of immunized mice was diluted and then incubated in Separate wells of tie apparatus. The blot was developed using an alkaline phosphatase-conjugated goat antibody to mouse IgG (Kirkegaard & Perry Labs, Gaithersburg, Md.) to identify the presence of BoNT/A-specific antibody.

Construction and expression of BoNT/A gene fragments. The BoNT/A gene was subcloned into overlapping fragments ranging in size from ˜300-600 base pairs by using PCR. The primers encoded flanking restriction sites that permitted convenient insertion into the expression vector used, and allowed transcriptional and translational read-through of the amplified fragments to occur (Table 1). Plasmid vector pMTD74 was used to express the amplified BoNT/A gene fragments in E. coli. Insertion of the PCR-amplified fragments into the expression vector pMTD74 resulted in translational fusion to the A2 peptide of cholera toxin (CtxA2) (8). The fragments were fused to CtxA2 to provide a vaccine for administration mucosally. The presence of BoNT/A-specific protein was determined by immunoblotting analysis, using polyclonal horse antiserum to BoNT/A, and by comparison of the predicted size of the truncated protein to its actual size. Fusion to CtxA2 increased the predicted size of the truncated BoNT/A proteins expressed by an additional 5.4 kDa, but it did not appear to affect their ability to be produced. By expressing overlapping segments of the toxin, all potential linear epitopes were encoded. BoNT/A is post translationally dleaved into the light (L) and heavy (H) chains which are joined together by a disulfide bond. The position of each fragment within BoNT/A is indicated by the chain it was derived from (L or H), followed by the amino acid residues of BoNT/A encoded.). The T7 promoter expressed these proteins at high levels in E. coli. The BoNT/A-specific proteins were expressed primarily in the form of inclusion bodies that could be isolated by differential centrifugation upon lysis of the cells.

Purification of BoNT/A proteins While the crude lysates containing the BoNT/A proteins were initially used for immunization of mice, it was found preferable to use at least partially purified materials to provide improved tolerance and to effectively produce strong, specific antibody response. For these reasons, the lysates containing the truncated BoNT/A proteins were subjected to purification by preparative SDS-PAGE and then used for immunization of mice. Preparative SDS-PAGE provided a convenient method of both solubilizing and separating the BoNT/A proteins from the majority of other contaminants present in the lysates. Although the BoNT/A proteins were not always purified to homogeneity, they were highly enriched. Furthermore, the BoNT/A proteins remained soluble after the SDS was removed, which facilitated the administration of these proteins to mice.

Immunogenicity of BoNT/A proteins. Mice were immunized i.p. with the truncated BoNT/A proteins emulsified in Ribi™ adjuvant. The mice were immunized at 2-week intervals, and one week after the last immunization, their serum was analyzed for the presence of antibody to BoNT/A. Since BoNT/A can be separated by SDS-PAGE into a 50 kDa light chain and 100 kDa heavy chain, immunoblotting analysis was used to evaluate whether the antibody elicited by each truncated protein reacted with the appropriate chain. Optimal antibody responses were observed in mice after the fourth dose. All of the truncated proteins were able to elicit an antibody response except H₁₀₇₈₋₁₂₂₀ Although this fragment was non-immunogenic, it was highly antigenic when reacted with polyclonal horse antiserum to BoNT/A. Unlike the crude lysates used for immunization previously, the purified proteins were well-tolerated arid could be repeatedly administered to the mice. In addition, the purified proteins were able to elicit an BoNT/A-specific antibody response in mice. This difference in the immunogenicity of the crude lysates cannot be accounted for by the lack of BoNT/A-specific protein, since the lysates used for immunization were known to contain appreciable quantities of truncated protein.

Protective efficacy of BoNT/A proteins. Two weeks after the final immunization, each mouse was challenged i.p. with 2 lethal doses of BoNT/A (2 MIPLD₉₉). This dose was chosen for initial screening to observe any potential ability of the proteins to elicit protective immunity. As shown in Table 3, only two proteins protected the majority of animals from death. Both of these fragments were derived from the heavy chain and encoded amino acid residues H₄₅₅₋₆₆₁ and H₁₁₅₀₋₁₂₈₉, H₄₅₅₋₆₆₁ of serotype A neurotoxin is the sequence

H₃N—IKVNN WDLFF SPSED NFTND LNKGE EITSD TNIEA
AEENI SLDLI QQYYL TFNFD NEPEN ISIEN LSSDI IGQLE
LMPNI ERFPN GKKYE LDKYT MFHYL RAQEF EHGKS RIALT
NSVNE ALLNP SRVYT FFSSD YVKKV NKATE AAMFL GWVEQ
LVYDF TDETS EVSTT DKIAD ITIII PYIGP ALNIG NMLYK
DDFVG ALIFS GA—COOH

and H₁₁₅₀₋₁₂₈₉ of serotype A neurotoxin is the sequence

H₃N—LNSSL YRGTK FIIKK YASGN KDNIV RNNDR VYINV
VVKNK EYRLA TNASQ AGVEK ILSAL EIPDV GNLSQ VVVMK
SKNDQ GITNK CKMNL QDNNG NDIGF IGFHQ FNNIA KLVAS
NWYNR QIERS SRTLG CSWEF IPVDD—COOH.

Although some of the other truncated proteins appeared to provide partial protection at the challenge dose initially used, none were as definitive as H₄₅₅₋₆₆₁ and H₁₁₅₀₋₁₂₈₉. Rechallenge of the survivors with 2 MIPLD₉₉ of BoNT/A resulted in the death of all mice except those immunized with the two protective fragments. To confirm these results, separate groups of mice were immunized with fragments H₄₅₅₋₆₆₁ and H₁₁₅₀₋₁₂₈₉ as before and then challenged with 10 MIPLD₅₀. The survival rate for mice immunized with H₄₅₅₋₆₆₁ and H₁₁₅₀₋₁₂₈₉ at this challenge dose was determined to be 87.5% and 60.0%, respectively.

TABLE 3
Immunogonicity and protective efficacy of the truncated BoNT/A proteins

| Protein | | | |
|---|---|---|---|
| Segment^{a} | Immuno- | Number of | |
| Survival | Blot^{b} | Survivors^{c} | % |
| L₄₋₁₂₈ | + | 1/10 | 10.0 |
| L₁₂₆₋₂₉₁ | + | 0/8 | 0.0 |
| L₂₆₇₋₄₆₅ | + | 0/9 | 0.0 |
| H₄₅₅₋₆₆₁ | + | 7/9 | 77.8 |
| H₆₈₀₋₈₀₈ | + | 0/5 | 0.0 |
| H₇₈₀₋₉₃₉ | + | 2/7 | 28.6 |
| H₉₁₅₋₁₀₅₉ | + | 0/8 | 0.0 |
| H₉₈₂₋₁₁₂₃ | + | 1/9 | 11.1 |
| H₁₀₉₈₋₁₂₂₀ | − | 0/5 | 0.0 |
| H₁₁₅₀₋₁₂₈₉ | + | 6/8 | 75.0 |

^{a}Amino acid residue number of the light (L) chain and the heavy (H) chain.
^{b}CB6F1 mice were immunized i.p. with four doses of each protein at 2-week intervals. One week after the last dose, the mice were bled and the serum was analyzed by immunoblot for the presence of antibody specific for BoNT/A.
^{c}Number of survivors/total number 4 days after challenge with 2 MIPLD₉₉ of BoNT/A.

Immunoblotting analysis was used to detect the presence of BoNT/A-specific antibody in the immunized mice for several reasons, First, the sensitivity of this method maximized the probability of detecting the presence of any fragment-specific antibody, regardless of whether it was directed towards a linear or a conformational epitope. Second, by separating BoNT/A into its heavy and light chains, this procedure also permitted the chain specificity or the antibody to be confirmed. By this method, all fragments were able to elicit an antibody response, except H_{1078 1220}.

Although most of the BoNT/A fragments were able to elicit antibody, only two were clearly able to confer protective immunity (Table 3). The protective efficacy of H₄₅₅₋₆₆₁ and H₁₁₅₀₋₁₂₈₉ correlates well with the potential functional role of these domains. The N-terminal half of the heavy chain (H_{N}) of BoNT/A, from which H₄₅₅₋₆₆₁. was derived, has been shown to be important in productive binding and internalization or the toxin to the cell. The C-terminal half of the heavy chain (H_{C}), from which H₁₁₅₀₋₁₂₈₉ was derived, has been associated with the initial binding of the toxin to the cell. If these functions are encoded by either fragment, then antibody specific to these domains would be predicted to interfere with the binding and/or internalization of BoNT/A. This, in turn, would prevent intoxication of the cell . The location of these protective domains on the extreme N- and C-terminal ends of the heavy chain suggest that important functional roles may also be encoded by these fragments. We are currently exploring this possibility.

The light chain fragment L₁₂₆₋₂₇₁ did not confer protection even though it elicited an antibody response (Table 3). This fragment encodes a highly conserved histidine-rich motif characteristic of zinc-dependent metalloproteases, such as BoNT/A. Although unproven, antibody directed to this region may block the enziymatic activity of BoNT/A, The inability of L₁₂₆₋₂₇₁ to protect suggests that the antibody elicited by this fragment may not have been directed towards epitopes involved in the enzymatic activity of the light chain.

Studies with MAbs suggest that many of the antibody determinants of BoNT/A may be conformationally sensitive, and there is evidence to suggest that BoNT/A is an oligomeric protein. If BoNT/A is indeed oligomeric, then it is possible that some epitopes are formed by the interaction of adjoining subunits. Alternatively, linear-distant parts of the toxin molecule may come together when folded to form epitopes, as appears to be the case for the light chain. Comparison of the amino acid sequence of these fragments with the amino acid Sequence of similar regions from the other serotypes did not show any significant homology. A cocktail of recombinant proteins containing amino acid sequences from analogous domains other serotypes (H₄₅₅₋₆₆₁ and H₁₁₅₀₋₁₂₈₉) should be prepared using the methods of the invention to provide immune protection against more than one serotype of organism.

The entire domains of H₄₅₅₋₆₁₁ and/or H₁₁₅₀₋₁₂₈₉ need not be used to provide a vaccine. However, at least 100 amino acids from one of the domains of any serotype should be used to provide sufficient antigenicity and immunoprotection.

What we claim is:

1. An isolated polypeptide chosen from those having at least 100 amino acids from domains H₄₅₅₋₆₆₁ and/or H₁₁₅₀₋₁₂₈₉ of the neurotoxin C. botulinum.

2. A composition of matter comprising at least one polypeptide of claim 1.

3. A polypeptide of claim 1 bound to a second peptide which acts as an adjuvant.

4. The polypeptide of claim 3 wherein the second peptide is A2 peptide of cholera toxin.

5. The composition of claim 2 wherein the polypeptide of neurotoxin of C botulinum is bound to the A2 peptide of cholera toxin.

6. A method of immunizing a mammal susceptible to botulism by administration of a composition of claim 3.

7. The method of claim 6 wherein the composition contains a polypeptide of neurotoxin of C. botulinum bound to the A2 peptide of cholera toxin.
